(19) United States
(12) Patent Application Publication
(10) Pub. No.: US 2007/0134355 A1
(43) Pub. Date: Jun. 14, 2007

(54) Use of plant parts of prickly pear (opuntia) and/or extracts therefrom for the treatment of depressions

(76) Inventors: Michael Nöldner, Karlsruhe (DE); Karl Schotz, Durmersheim (DE)

Correspondence Address: EDWARDS ANGELL PALMER & DODGE LLP, P.O. BOX 55874, BOSTON, MA, 02205, US

(21) Appl. No.: 10/577,624

(22) PCT Filed: Oct. 26, 2004
(86) PCT No.: PCT/EP04/12070

§ 371 Date: Apr. 28, 2006

(30) Foreign Application Priority Data
Oct. 28, 2003 (DE) 103 50 194.0

(51) Int. Cl. A61K 36/33

(52) U.S. Cl. 424/767

(57) ABSTRACT

The present invention relates to the use of Opuntias, plant parts thereof and/or extracts or other preparations produced therefrom for the treatment of episodes of depression and depressive diseases or other affective disorders, which can be influenced by antidepressive agents, such as anxiety or panic disorders, bipolar depressions, somatization disorders and the premenstrual syndrome, as well as preliminary stages of such diseases.

The present invention relates to the use of Opuntias, plant parts thereof and/or extracts or other preparations produced therefrom for the treatment of episodes of depression and depressive diseases or other affective disorders which can be influenced by antidepressive agents such as anxiety and panic disorders, bipolar depressions, somatization disorders and the premenstrual syndrome as well as preliminary stages of such diseases.

With a one year prevalence rate of about 10% and a lifetime prevalence rate of about 20% depressions are among the most frequent diseases in western industrial nations. They significantly affect the patient in the private and professional area, irritate his environment, enormously burden our health care system by an increased utilization of the primary medical care and by many lost working days due to sick certificates and are even mortal in specific cases. Estimations today assume that more than two third of the about 10,000 suicides per year in Germany trace back to depressions. Depressions are clinically categorized as follows (H.-J. Möller, Der Internist 41, 70-79 (2000)):

1. Endogenous depressions, unipolar and bipolar

2. Depressive neurosis

3. Reactive depression

4. Depression in case of schizoaffective psychosis

5. Organically-based depression

6. Depressive symptoms in dementia

Furthermore, depressive diseases are classified on the basis of their degree of severity into low, moderate or severe.

Preliminary stages of depressive diseases can manifest in abjectness, listlessness, melancholia, anergy, labile or saddened mood or limitations of the emotional well-being.

Despite a plurality of different hypotheses, the causes of depressive diseases are elucidated only insufficiently. For a medicamentous treatment of depression and other affective disorders, for example, the following medicament groups are suitable (H.-J. Möller, Der Internist 41, 70-79 (2000)):

1. Selective serotonin reuptake inhibitors (SSRI)

2. Selective noradrenaline reuptake inhibitors (SNRI)

3. Selective serotonin and noradrenaline reuptake inhibitors (SSNRI)

4. Tricyclic antidepressive agents (TCA)

5. MAO-A inhibitors

6. Other synthetic preparations

7. Phytopharmaca

It is a common feature of all the treatment methods mentioned hereinabove that, if there is an effect at all, the effect occurs after a treatment period of 10-14 days. For about 20% of the patients the medicamentous therapy is insufficient also in the case of a combination of different preparations. Furthermore, all of the treatment methods mentioned have the drawback that undesired side effects are caused which often lead to a withdrawal of the therapy. Therefore, there is a significant demand to provide further agents for the treatment of depressive diseases and other affective disorders and preliminary stages thereof, particularly those agents which totally or partially overcome one or more of the drawbacks mentioned above.

Thus, it is the object of the present invention to provide such agents.

According to the present invention, this object is solved by the use of Opuntias, plant parts thereof and/or extracts or other preparations produced therefrom, preferably from the flowers of Opuntias and particularly preferred from the flowers of the species Opuntia ficus-indica, for the treatment of episodes of depression and depressive diseases or other affective disorders which can be influenced by antidepressive agents, such as anxiety and panic disorders, bipolar depressions, somatization disorders and the premenstrual syndrome as well as preliminary stages of such diseases, as well as by a medicament and a dietetic food product for treating or for supporting the treatment of episodes of depression and depressive diseases or other affective disorders and preliminary stages thereof, characterized by a content of Opuntias, plant parts thereof and/or extracts or other preparations produced therefrom, as well as by a pharmaceutical preparation as an oral administration form which further contains suitable pharmaceutically acceptable adjuvants.

Extracts from the flowers of Opuntias are used for the treatment of diarrhea, hyperplasia of the prostate and colitis (British Herbal Pharmacopoiea 1983 published by the British Herbal Medicine Association's, p. 151-152). Extracts from the leaves and leaf sprouts (so-called Nopal) have a blood sugar-lowering effect in human beings (A. C. Frati-Munari et al., Diabetis Care, 63-66 (1988) and A. C. Frati-Munari et al., Gac. Med. Mex. 128, 431-436 (1992)). In pharmacological experiments effects have also been found for plant preparations produced in different ways. In particular, alcoholic extracts from the trunk exhibit anti-phlogistic, analgetic and antioxidative effects (Park et al., Arch. Pharm. Res. 21, 30-34 (1998) and Fitoterapia 72, 288-290 (2001) and Lee et al., J. Agric. Food Chem. 50, 6490-6496 (2002)). Furthermore, diuretic and anti-ulcer effects were demonstrated in rats (E. M. Galati et al., J. Ethnopharmacol. 79, 17-21 (2002) and E. M. Galati et al., J. Ethnopharmacol. 76, 1-9 (2001)). MAO-B-inhibiting effects were shown for the fruits of Opuntia ficus-indica var. saboten (Han et al., Arch. Pharm. Res. 24, 51-54 (2001)). Furthermore, neuroprotective properties of Opuntia ficus-indica extracts are described (Y. S. Lee et al., Korea Institute of Science and Technology, WO 03/037324).

It has surprisingly been found that Opuntias exhibit a significant antidepressive effect in animal experiments. Up to now such an effect has not been described for Opuntias and could not be expected on the basis of the so far known pharmacologic and clinical effects. Furthermore, it has surprisingly been noticed that Opuntias potentiate the antidepressive efficacy of venlafaxine in animal experiments. Venlafaxine represents the prototype for the last generation of antidepressive agents (so-called SSNRIs).

Extracts from Opuntias can be obtained according to known preparation methods in variable compositions using solvents such as water, methanol, ethanol, acetone and the like as well as mixtures thereof at temperatures from room temperature to 100° C. under slight to vigorous mixing within ten minutes to 24 hours under normal pressure to pressures of up to 200 bar. In order to enrich the active ingredients, further concentration steps can be carried out, such as liquid-liquid distribution using, for example, 1-butanol/water or ethylacetate/water, adsorption-desorption on ion exchangers, LH20, HP20 and other resins or chromatographic separations over RP18, silica gel and the like. If further processing to dry extracts is desired, this is carried out according to methods known per se by removing the solvent at increased temperature and/or reduced pressure.

The plant material according to the present invention and the extracts produced therefrom can be administered in the form of powders, granules, tablets, dragées (coated tablets) or capsules or also as a solution (such as tea, decoction), preferably orally.

For the production of tablets, the extract is mixed with suitable pharmaceutically acceptable adjuvants such as lactose, cellulose, silicon dioxide, croscarmellose and magnesium stearate and pressed into tablets which are optionally provided with a suitable coating, for example made of hydroxymethylpropylcellulose, polyethyleneglycol, dyes (such as titanium dioxide and iron oxide) and talcum.

The plant material according to the present invention and the extracts produced therefrom can also be filled into capsules, optionally under the addition of adjuvants such as stabilizers, fillers and the like. The dosage is carried out in such a way that 5 to 2,000 mg, preferably 10 to 1,000 mg and particularly preferred 50 to 500 mg of the extract or 50 mg to 10 g, preferably 0.5 to 5 g of the dried plant material are administered per day.

In the case of fresh (not dried or partially dried) plant material, the amount indicated above is based on the dry portion.

The efficacy of Opuntias against depressive diseases is confirmed by the experiments described in the following:

The antidepressive efficacy has been tested by means of the so-called “forced swimming test” in rats. In this test rats are brought into an impasse during a defined period of five minutes (glass cylinder filled with water). In this test the rats reacted with a rigour referred to as immobilization time, which is interpreted to correlate with a depression. If the rats are treated with an antidepressively effective medicament prior to the test, the immobilization time decreases. Since other psychopharamaca such as anxiolytics or neuroleptics are not efficacious in this test, this test system is well suitable to detect antidepressive effects (R. D. Porsolt et al., Eur. J. Pharmacol. 47, 379-391 (1978); R. D. Porsolt et al., Adv. Pharmacol. Sci., 137-159 (1991)). In order to be efficacious, each of the heretofore known antidepressive agents has to be administered over a period of several days in this test, like in the case of patients. The test animals were treated either with the test substance or with a solvent only for control purposes or with the tricyclic antidepressive agent Imipramine in order to compare the efficacy. Imipramine was selected as the standard comparative substance, because it is one of the strongest antidepressive agents both in psychiatric practice and in animal model.

Tables 1 to 3 exemplarily show the efficacy of three different Opuntia extracts compared to the tricyclic antidepressive agent Imipramine as determined after a treatment period of 9 days. Furthermore, the venlafaxine-potentiating effect of Opuntia extract is demonstrated in Table 4. In this case, the inhibition was determined already after a treatment period of 3 days. For comparison purposes, the inhibition of the immobility was represented as the percentage of inhibition against the control group in each of the tables.

TABLE 1

| | Dose | Inhibition of immobility |
|---|---|---|
| Substance | [mg/kg] | [%] |
| Opuntia extract (Example 1) | 10 | 16 |
| Opuntia extract (Example 1) | 30 | 34 |
| Opuntia extract (Example 1) | 100 | 52 |
| Opuntia extract (Example 1) | 300 | 83 |
| Imipramine | 30 | 64 |

TABLE 2

| | Dose | Inhibition of immobility |
|---|---|---|
| Substance | [mg/kg] | [%] |
| Opuntia extract (Example 2) | 30 | 14 |
| Opuntia extract (Example 2) | 100 | 33 |
| Opuntia extract (Example 2) | 300 | 65 |
| Imipramine | 30 | 68 |

TABLE 3

| | Dose | Inhibition of immobility |
|---|---|---|
| Substance | [mg/kg] | [%] |
| Opuntia extract (Example 3) | 100 | 23 |
| Imipramine | 20 | 44 |

TABLE 4

| | Dose | Inhibition of immobility |
|---|---|---|
| Substance | [mg/kg] | [%] |
| Opuntia extract (Example 1) | 200 | 43 |
| Venlafaxine | 100 | 25 |
| Opuntia extract (Example 1) + | 200 | 61 |
| Venlafaxine | 100 | |

### EXAMPLE 1

### Dry Extract (Extraction Solvent: 60% by Weight EtOH) from the Flowers of Opuntia ficus-indica

200 g of finely ground drug were stirred twice for 1 h at 60° C. using 1400 g 60% by weight EtOH, respectively. Subsequently, the suspension is filtered with suction over a glass frit P4, the combined filtrates are set free from EtOH at 40° C. in vacuum, the remaining aqueous residue is frozen and lyophilized. The solid obtained is dried in vacuum at 40° C. over P₂O₅ and KOH: 30.2 g (15.1%) of dry extract.

### EXAMPLE 2

### Dry Extract (Aqueous Extract) from the Flowers of Opuntia ficus-indica (Decoction)

200 g of ground flowers are doused with 1.5 l of boiling water, stirred for a short time and left for 10 minutes. The voluminous mass is subsequently filtered over glass fritt P4, the solid is again doused with 1 l of boiling water, the mass is filtered with suction over a glass frit P4 after 10 minutes, the remaining voluminous residue is centrifugated, the decanted solution is combined with the filtrates and freeze-dried. Subsequently, the residue is ground and dried in vacuum for 24 h: 23.4 g (11.7%).

### EXAMPLE 3

### Dry extract (extraction solvent: 96% by weight EtOH) from young leaf sprouts of Opuntia ficus-indica

12.0 kg fresh young leaf sprouts of Opuntia ficus-indica are reduced to small pieces and added with 16 kg of 96% ethanol. The mixture is homogenized for 10 minutes at room temperature using an ultraturrax, set free from coarse plant material using a suction filter and the turbid filtrate is filtered over a Seitz filter 1500. The clear solution is then concentrated at 40° C. in vacuum to yield the spissum extract and the viscous mass is dried in a drying cabinet at 40° C. in vacuum: 248 g (2.1%, corresponding to 13.8%, calculated on the basis of a dry content of the plant material of 15%).

### EXAMPLE 4

### Tablets

A dry extract of Opuntia ficus-indica (Example 1) is mixed with adjuvants and pressed into tablets (core of the tablets=position 1-6). The tablets are provided with a coating of hydroxypropylmethylcellulose (position 7-10).

| | Ingredient | mg/tablet |
|---|---|---|
| 1 | Dry extract from Opuntia ficus-indica | 100.0 |
| 2 | Microcrystalline cellulose | 117.0 |
| 3 | Lactose monohydrate | 58.0 |
| 4 | Croscarmellose | 15.0 |
| 5 | Highly dispersed silicon dioxide | 3.0 |
| 6 | Magnesium stearate | 6.0 |
| 7 | Hydroxypropylmethylcellulose | 15.0 |
| 8 | Polyethyleneglycol | 3.0 |
| 9 | Talcum | 1.0 |
| 10 | Titanium dioxide | 2.0 |

1-22. (canceled)

23. A method for treating a subject suffering from or susceptible depression, depressive diseases or other affective disorder which can be influenced by an antidepressive agent, comprising administering to the subject Opuntias, plant parts thereof and/or extracts or other preparations produced therefrom.

24. The method of claim 23 wherein the subject is suffering from depression, a depressive disease or other affective disorder which can be influenced by an antidepressive agent.

25. The method of claim 23 the subject is suffering from or susceptible to anxiety or panic disorder, bipolar depression, somatization disorder or premenstrual syndrome.

26. The method of claim 23 wherein the subject is suffering from a disease or disorder having a preliminary stage that includes abjectness, listlessness, melancholia, anergy, labile or saddened mood or limitations of emotional well-being.

27. The method of claim 23 wherein the subject has been identified as suffering from or susceptible to depression, depressive disease or other affective disorder which can be influenced by an antidepressive agent, and Opuntias, plant parts thereof and/or extracts or other preparations produced therefrom is administered to the identified subject.

28. The method of claim 23 wherein the subject has been identified as suffering from depression, depressive disease or other affective disorder which can be influenced by an antidepressive agent, and Opuntias, plant parts thereof and/or extracts or other preparations produced therefrom is administered to the identified subject.

29. The method of claim 23 wherein plant parts are administered to the subject.

30. The method of claim 29 wherein the plant parts are flowers and/or leaf sprouts of Opuntias.

31. The method of claim 23 wherein the Opuntias belong to the species Opuntia ficus-indica.

32. The method of claim 23 wherein extracts from Opuntias or plant parts thereof are administered to the subject.

33. A pharmaceutical composition comprising Opuntias, plant parts thereof and/or extracts or other preparations produced therefrom.

34. The pharmaceutical composition of claim 33 further comprising one or more selective serotonin and noradrenaline reuptake inhibitors (SSNRI).

35. The pharmaceutical composition of claim 33 further comprising venlafaxine.

36. The pharmaceutical composition of claim 33 further comprising one or more suitable adjuvants.

37. The pharmaceutical composition of claim 33 wherein the pharmaceutical composition is in an oral administration form.

38. A dietetic food product comprising Opuntias, plant parts thereof and/or extracts or other preparations produced therefrom.
